(19) United States
(12) Patent Application Publication
(10) Pub. No.: US 2011/0202121 A1
(43) Pub. Date: Aug. 18, 2011

(54) ELECTRICAL NERVE STIMULATOR

(76) Inventor: SHIN-HENG WEN, Kaohsiung City (TW)

(21) Appl. No.: 12/706,546

(22) Filed: Feb. 16, 2010

(51) Int. Cl. A61N 1/04

(52) U.S. Cl. 607/145

(57) ABSTRACT

An electrical nerve stimulator includes a main probe device receiving a current signal generated by a current generator. The main probe device includes a probe disposed in a housing and having a tip extending outwardly of a tubular end portion of the housing, and a cover mounted detachably on the end portion of the housing and having a flexible absorbent cap portion for covering the tip of the probe. The main probe device is operable so as to generate a stimulating signal based on the current signal from the current generator and to output the stimulating signal through the probe.

### BACKGROUND OF THE INVENTION

1. Field of the Invention

The invention relates to a physical therapy device, more particularly to an electrical nerve stimulator.

2. Description of the Related Art

FIG. 1 illustrates a conventional nerve ending therapeutical device disclosed in U.S. Pat. No. 4,848,357. The conventional nerve ending therapeutical device includes a current generator 11 for generating a current signal, a probe 12 connected electrically to the current generator 11 for receiving the current signal therefrom and outputting the current signal, and a handle 13 connected electrically to the current generator 11. In use, a tip 121 of the probe 12 is pressed on the skin of a human body of a user, and the handle 13 is grasped by one hand of the user. As a result, the current signal can flow through the circulatory system of the human body so as to stimulate the internal secretion of the same, thereby providing therapeutical effects.

However, in actual use, the tip 121 of the probe 12 does not directly contact the skin of the human body for hygiene purposes. As shown in FIG. 2, to ensure electrical conduction between the tip 121 and the skin of the human body, an absorbent member 14 serving as a cover, such as a sheet of tissue, is fixed by a fastener 15, such as a rubber band, to the probe 12 so as to cover the tip 121, and is then soaked in liquid, such as medicinal liquid, to thereby achieve electrical conduction between the tip 121 and the skin of the human body. Therefore, replacement of the absorbent member 14 is required prior to use of the probe 12 by another user, thereby resulting in inconvenience during use.

### SUMMARY OF THE INVENTION

Therefore, an object of the present invention is to provide an electrical nerve stimulator that can overcome the aforesaid drawbacks of the prior art.

According to the present invention, an electrical nerve stimulator comprises:

a current generator for generating a current signal; and

a main probe device connected electrically to the current generator for receiving the current signal therefrom, and including
a housing having a tubular end portion,
a probe disposed in the housing, and having a tip extending outwardly of the end portion of the housing, and
a cover mounted detachably on the end portion of the housing and having a flexible absorbent cap portion for covering the tip of the probe.

The main probe device is operable so as to generate a stimulating signal based on the current signal from the current generator and to output the stimulating signal through the probe.

### BRIEF DESCRIPTION OF THE DRAWINGS

Other features and advantages of the present invention will become apparent in the following detailed description of the preferred embodiments with reference to the accompanying drawings, of which:

FIG. 1 is a perspective view of a conventional nerve ending therapeutical device;

FIG. 2 is a perspective view showing a probe of the conventional nerve ending therapeutical device when in a state of use;

FIG. 3 is a perspective view showing the first preferred embodiment of an electrical nerve stimulator according to the present invention;

FIG. 4 is a schematic electrical block diagram illustrating the first preferred embodiment;

FIG. 5 is a fragmentary, partly schematic sectional view showing a main probe device of the first preferred embodiment;

FIG. 6 is a schematic view of the main probe device of the first preferred embodiment illustrating how a cover is moved away from a tubular end portion of a housing;

FIG. 7 is a fragmentary exploded perspective view showing a first variation of the main probe device of the first preferred embodiment;

FIGS. 8 and 9 are fragmentary schematic views showing a second variation of the main probe device of the first preferred embodiment;

FIG. 10 is a fragmentary schematic view of the second variation of the main probe device of the first preferred embodiment illustrating how the cover is moved away from the tubular end portion of the housing;

FIG. 11 is a schematic view showing the first preferred embodiment when in a state of use;

FIG. 12 is a schematic electrical block diagram illustrating the second preferred embodiment of an electrical nerve stimulator according to the present invention;

FIG. 13 is a perspective view showing a main probe device of the second preferred embodiment;

FIG. 14 is a partly exploded perspective view showing a variation of the main probe device of the second preferred embodiment;

FIG. 15 is a schematic electrical block diagram illustrating the third preferred embodiment of an electrical nerve stimulator according to the present invention;

FIG. 16 is a perspective view showing the third preferred embodiment; and

FIG. 17 is a perspective view showing the third preferred embodiment when in a state of use.

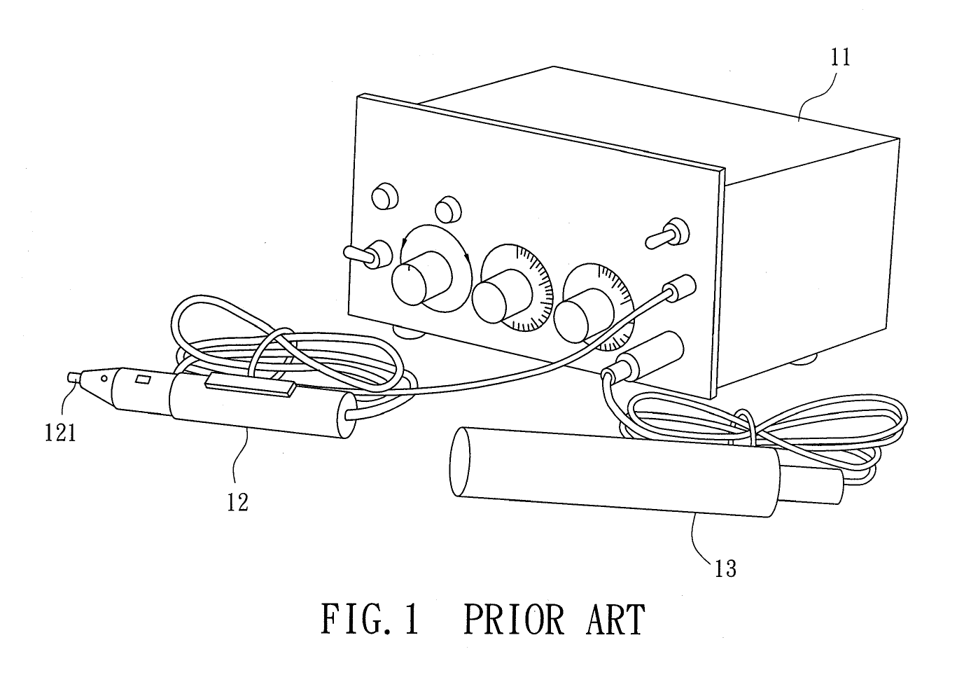

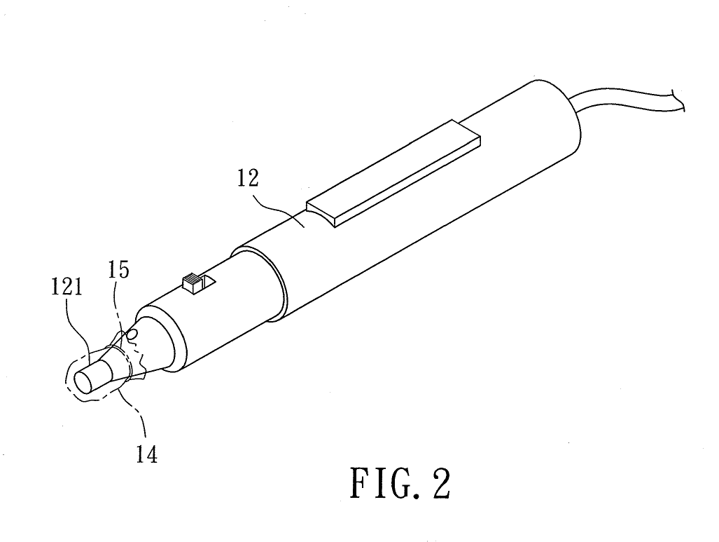

### DETAILED DESCRIPTION OF THE PREFERRED EMBODIMENTS

Before the present invention is described in greater detail, it should be noted that like elements are denoted by the same reference numerals throughout the disclosure.

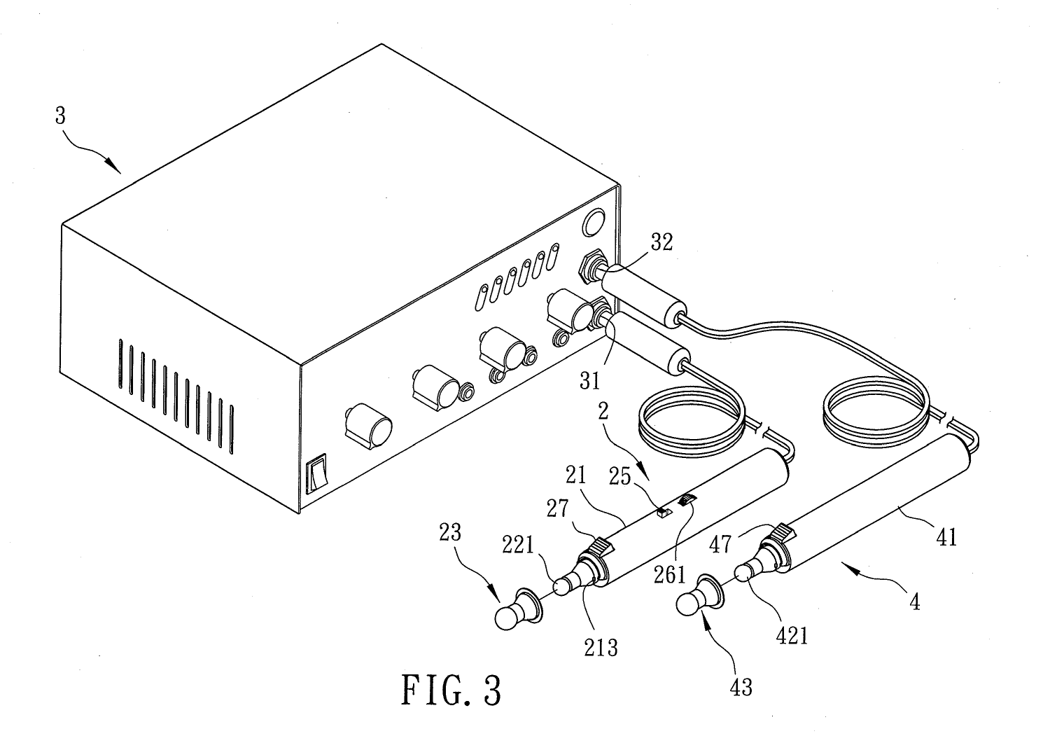

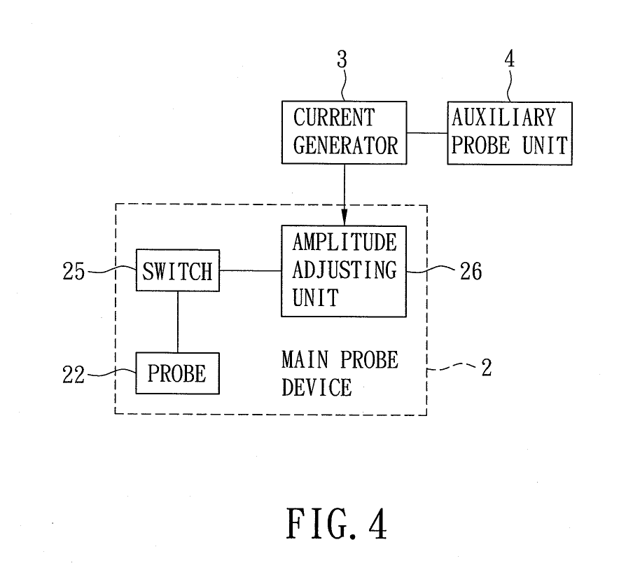

Referring to FIGS. 3 and 4, the first preferred embodiment of an electrical nerve stimulator according to the present invention is shown to include a current generator 3, a main probe device 2, and an auxiliary probe unit 4.

In this embodiment, the current generator 3 is operable so as to generate a current signal through an output port 31.

The main probe device 2 includes a housing 21, a probe 22, a cover 23, an actuator 27, an amplitude adjusting unit 26, and a switch 25.

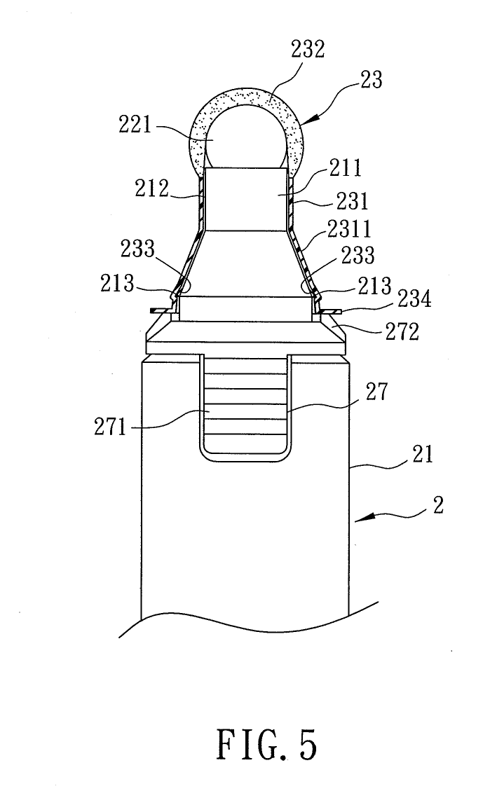

The housing 21 has a tubular end portion 211. In this embodiment, as shown in FIG. 5, the end portion 211 is defined by an annular wall that has an annular outer surface 212 formed with two protrusions 213 opposite to each other.

The probe 22 is disposed in the housing 21, and has a tip 221 extending outwardly of the end portion 211 of the housing 21.

The cover 23 is mounted detachably on the end portion 211 of the housing 21, and has a sleeve portion 231 sleeved on the end portion 211 of the housing 21, and a flexible absorbent cap portion 232 connected to the sleeve portion 231 for covering the tip 221 of the probe 22. In this embodiment, as shown in FIG. 5, the sleeve portion 231 has a rim flange 234, and an annular inner surface 2311 formed with two first positioning grooves 233 that engage respectively and releasably the protrusions 213 of the end portion 211 of the housing 21, thereby positioning the cover 23 on the end portion 211 of the housing 21. The absorbent cap portion 232 is made of a cotton material.

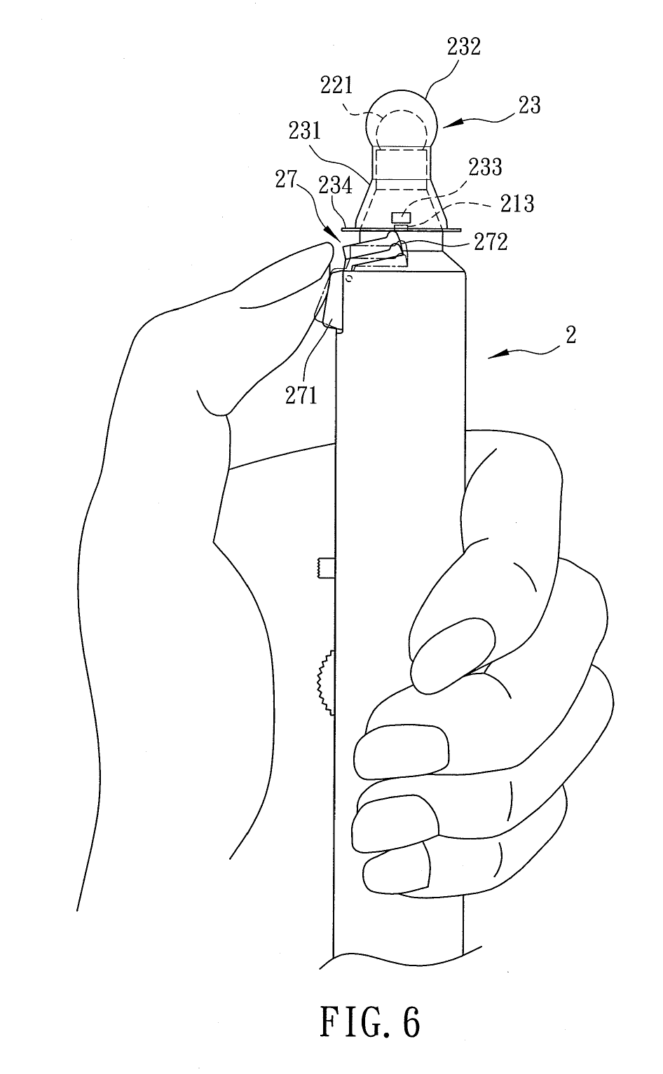

The actuator 27 is mounted movably on the housing 21, and has an operating end portion 271, and a pushing end portion 272 opposite to the operating end portion 271. As shown in FIG. 6, the pushing end portion 272 pushes the rim flange 234 of the sleeve portion 231 of the cover 23 in response to movement of the operating end portion 271 to move the cover 23 away from the end portion 211 of the housing 21 such that the protrusions 213 of the end portion 211 of the housing 21 disengage respectively the first positioning grooves 233 in the sleeve portion 231 of the cover 23.

The amplitude adjusting unit 26 is connected electrically to the current generator 3 for receiving the current signal therefrom. In this embodiment, the amplitude adjusting unit 26 is wiredly-connected to the output port 31 of the current generator 3. The amplitude adjusting unit 26 includes a control key 261 serving as a control member and mounted on the housing 21, and adjusts amplitude of the current signal in response to operation of the control key 261 so as to generate a stimulating signal.

The switch 25 is mounted on the housing 21 and is connected electrically between the amplitude adjusting unit 26 and the probe 22. The switch 25 is operable between an ON-state, where the stimulating signal is outputted from the amplitude adjusting unit 26 through the switch 25 and the probe 22, and an OFF-state, where output of the stimulating signal is terminated.

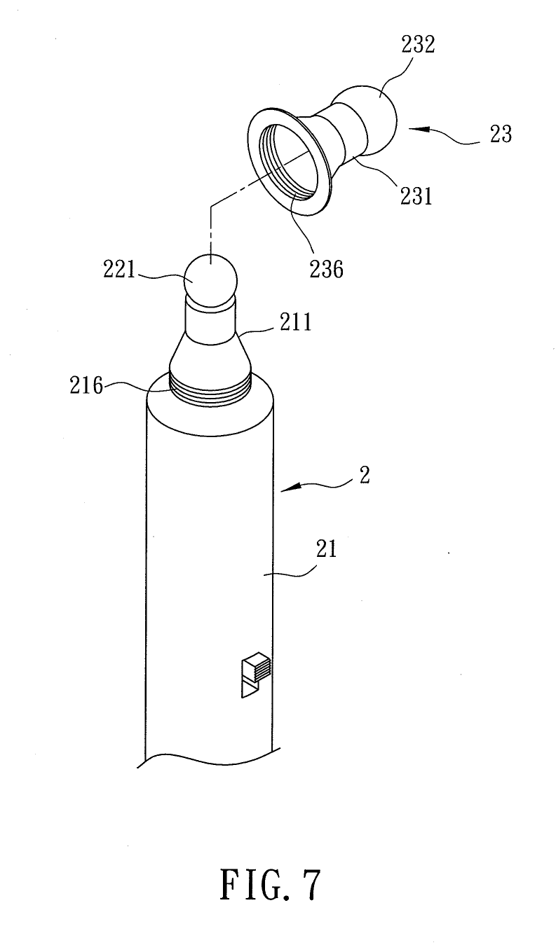

FIG. 7 illustrates a first variation of the main probe device 2, wherein the actuator 27, the first positioning grooves 233 in the sleeve portion 231, and the protrusions 213 of the end portion 211 of the housing 21 are omitted. It is noted that the end portion 211 has an outer threaded section 216, and that the sleeve portion 231 has an inner threaded section 236 engaging releasably the outer threaded section 216. Thus, the cover 23 is connected threadedly to the end portion 211 of the housing 21.

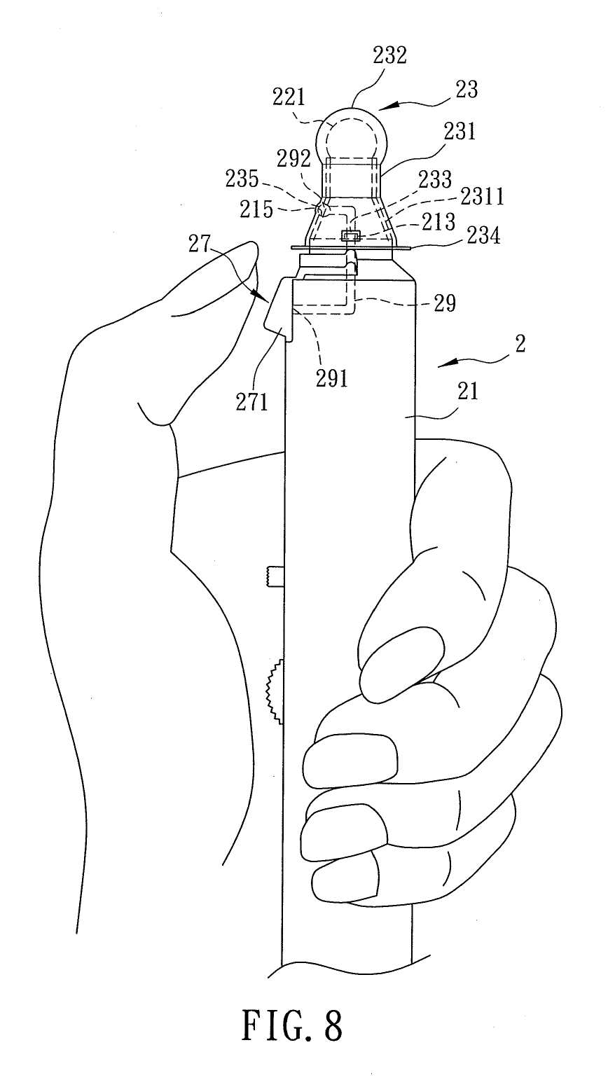

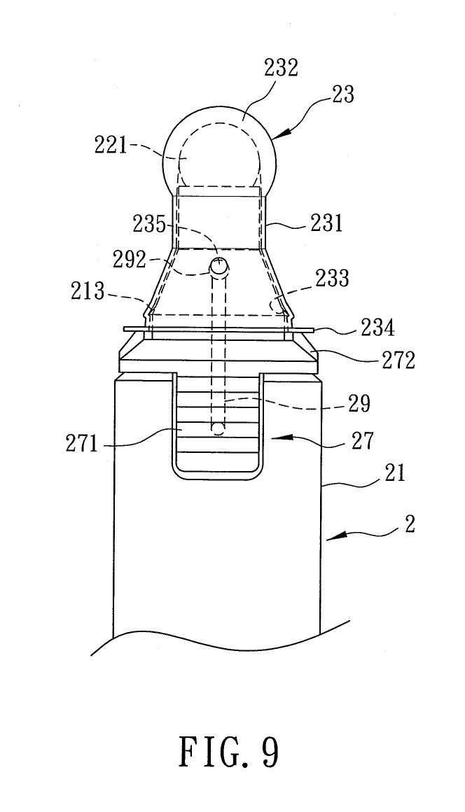

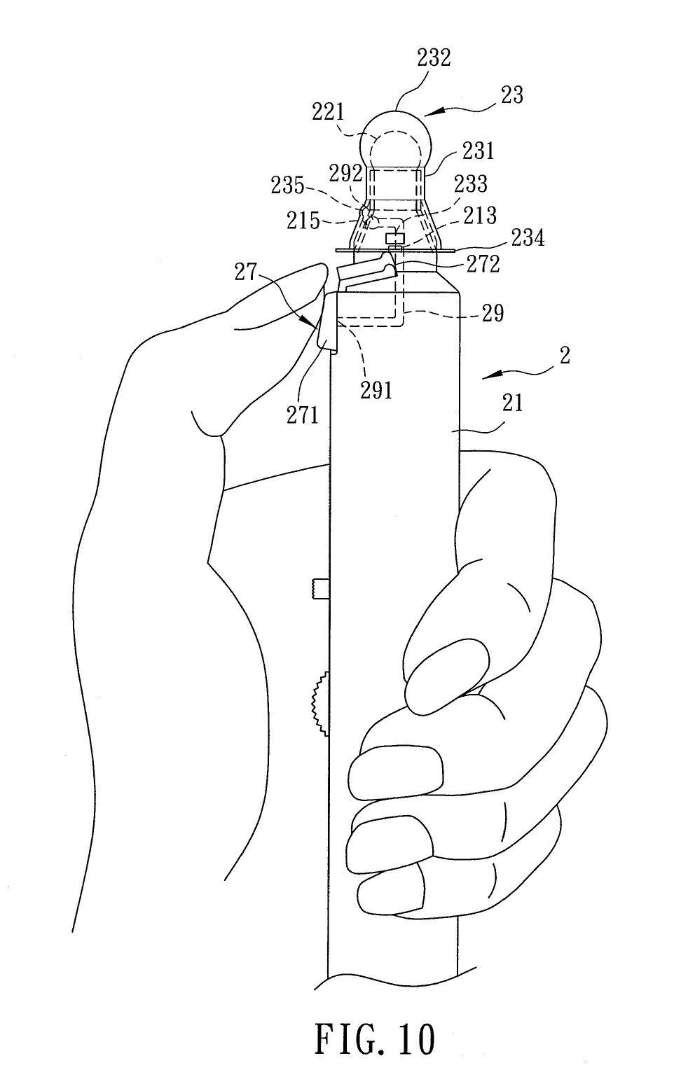

FIGS. 8 and 9 illustrate a second variation of the main probe device 2, wherein the annular inner surface 2311 of the sleeve portion 231 of the cover 23 is further formed with a second positioning groove 235. The annular wall defining the end portion 211 of the housing 21 is formed with a through hole 215 corresponding to the second positioning groove 235. The main probe device 2 further includes a driving rod 29 disposed in the housing 21. The driving rod 29 has a connecting end 291 connected fixedly to the operating end portion 271 of the actuator 27 such that the driving rod 29 is movable with the operating end portion 271 of the actuator 27, and an engaging end 292 opposite to the connecting end 291, extending outwardly of the through hole 215 in the end portion 211 of the housing 21 and engaging releasably the second positioning groove 235 in the sleeve portion 231 of the cover 23. The engaging end 292 moves away from the second positioning groove 235 in response to movement of the operating end portion 271 of the actuator 27 to thereby disengage the second positioning groove 235 in the sleeve portion 231 of the cover 23, as shown in FIG. 10.

In this embodiment, the auxiliary probe unit 4 is connected wiredly and detachably to an input port 32 of the current generator 3 (see FIG. 3), and includes a handle body 41, a probe (not shown) disposed in the handle body 41 and having a tip 421 extending outwardly of the end portion 411 of the handle body, and a cover 43 and an actuator 47. It is noted that, since the end portion 411 of the handle body 41, the cover 43 and the actuator 47 have the same configurations and operations as those of the end portion 211 of the housing 21, the cover 23 and the actuator 27 of the main probe device 2, details of the same are omitted herein for the sake of brevity.

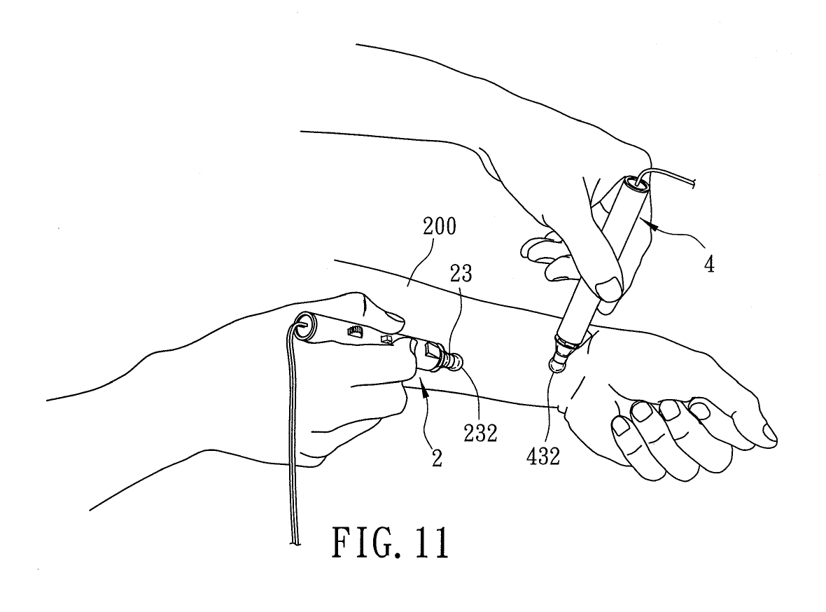

In use, as shown in FIG. 11, the absorbent cap portions 232, 432 of the covers 23, 43 of the main probe device 2 and the auxiliary probe unit 4 soak in liquid, such as medicinal liquid, and contact the skin of a human body 200 at first and second positions, such as two different acupuncture points such that the current generator 3, the main probe device 2, the human body and the auxiliary probe unit 4 constitute a closed loop. As such, the stimulating signal is outputted to the human body 200 through the tip 221 of the probe 22 of the main probe device 2 for physical therapy. It is noted that an impedance between the first and second positions of the skin of the human body can be obtained such that circulatory condition of the human body can be realized based on the impedance.

In sum, since the switch 25 is mounted on the housing 21 of the main probe device 2, the user who holds the main probe device 2 can easily control output of the stimulating signal. Furthermore, for each of the main probe device 2 and the auxiliary probe unit 3, due to the presence of the protrusions 213, the positioning grooves 233, and the actuator 27, 47, replacement of the cover 23, 43 is easier as compared with the aforesaid prior art, thereby resulting in convenience during use.

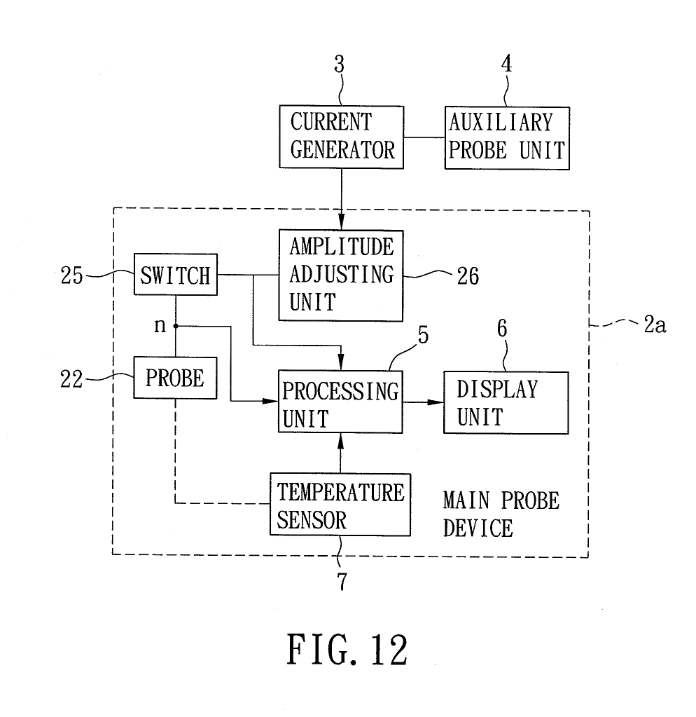

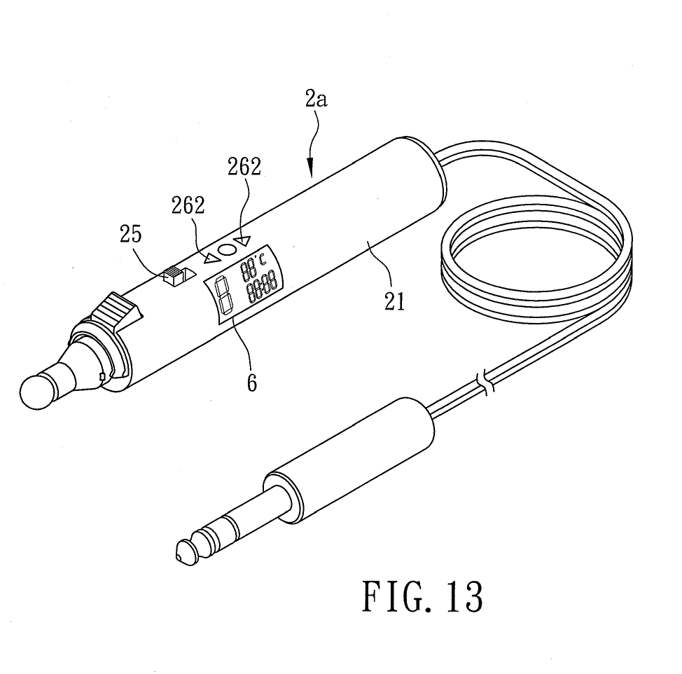

FIGS. 12 and 13 illustrate the second preferred embodiment of an electrical nerve stimulator according to this invention, which is a modification of the first preferred embodiment.

In this embodiment, the control member of the amplitude adjusting unit 26 of the main probe device (2a) includes two control keys 262 mounted on the housing 21. Furthermore, the main probe device (2a) further includes a processing unit 5, a temperature sensor 7, and a display unit 6.

The processing unit 5 is disposed in the housing 21, is coupled to the amplitude adjusting unit 26 and to a common node (n) between the switch 25 and the probe 22, receives the stimulating signal from the amplitude adjusting unit 26 so as to generate amplitude information associated with the stimulating signal, and calculates duration of the ON-state of the switch 25, i.e., therapy duration, based on detection of output at the common node (n) so as to generate therapy duration information associated with the duration of the ON-state of the switch 25.

The temperature sensor 7 is disposed in the housing 21, is coupled to the processing unit 5, and senses temperature of the probe 22 so as to output an output signal associated with the temperature of the probe 22 to the processing unit 5. The processing unit 5 receives the output signal from the temperature sensor 7 so as to generate temperature information corresponding to the temperature of the probe 22.

The display unit 6 is mounted on the housing 21, and is coupled to the processing unit 5 for displaying the amplitude information, the therapy duration information and the temperature information thereon.

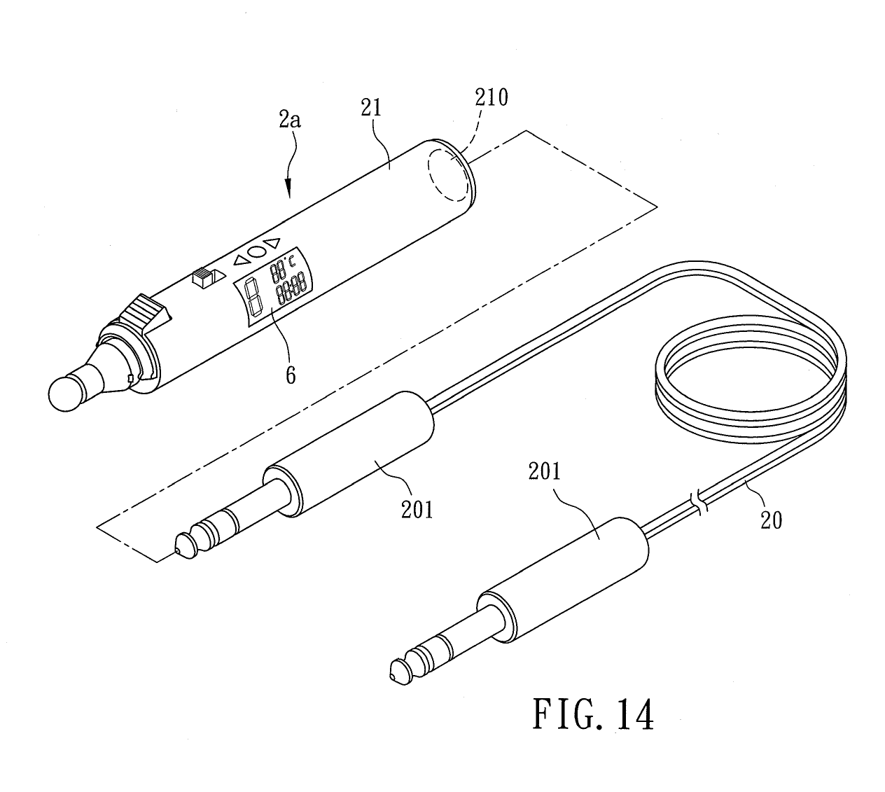

FIG. 14 illustrates a variation of the main probe device (2a), wherein the main probe device (2a) further includes a separable connecting wire 20 having opposite plugs 201 coupled respectively and detachably to a socket 210 mounted on the housing 21, and the output port 31 of the current generator 3 (see FIG. 3) during use.

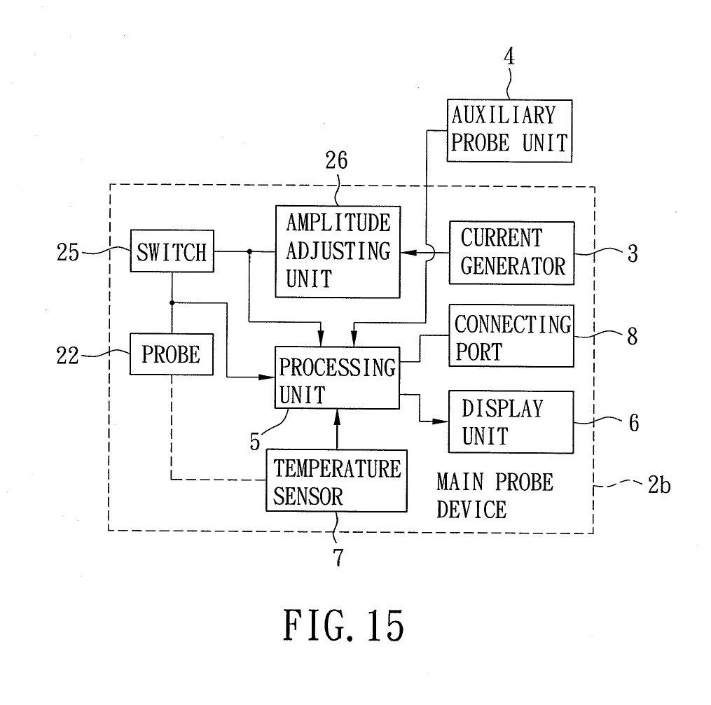

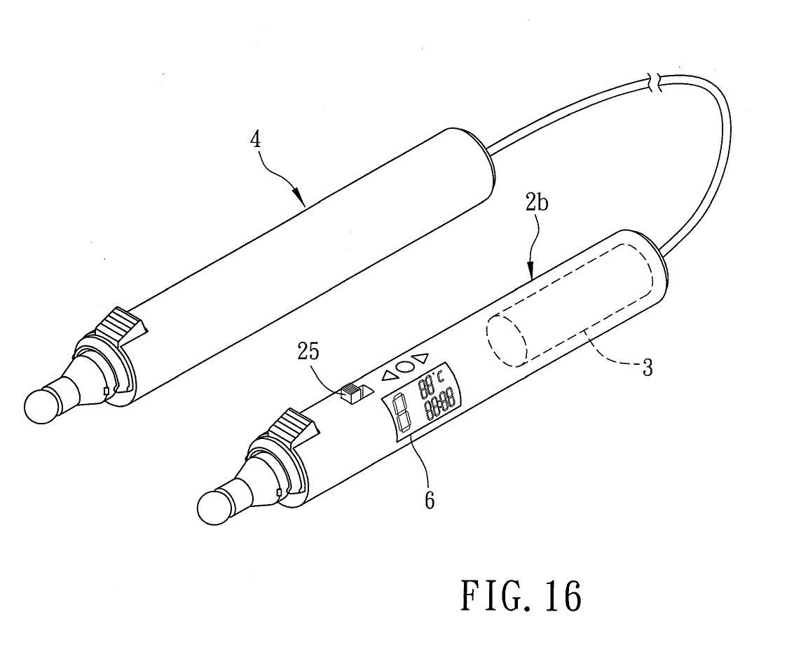

FIGS. 15 and 16 illustrate the third preferred embodiment of an electrical nerve stimulator according to this invention, which is a modification of the second preferred embodiment.

In this embodiment, the main probe device (2b) includes the current generator 3. The current generator 3 is disposed in the housing 21. It is noted that the auxiliary probe unit 4 is wiredly-connected to the processing unit 5 of the main probe device (2b). As a result, the processing unit 5 further calculates the impedance between the first and second positions where the main probe device (2b) and the auxiliary probe unit 4 respectively contact the skin during use so as to generate circulatory information corresponding to the user based on the impedance. Therefore, in such a configuration, the electrical nerve stimulator of this embodiment is portable.

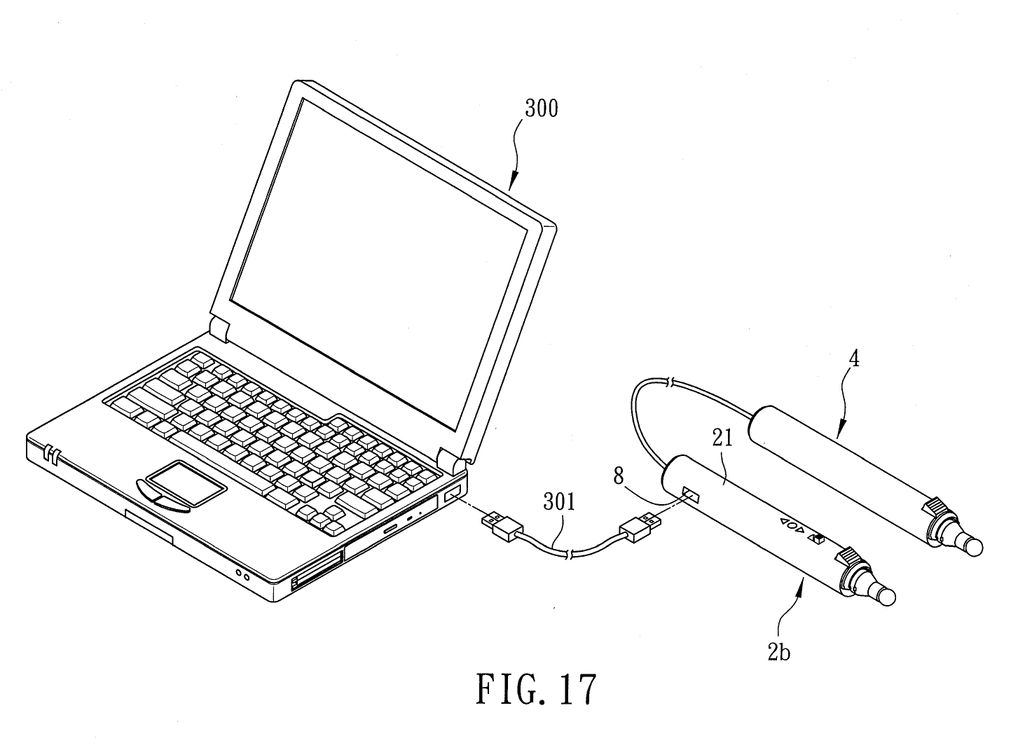

Furthermore, the main probe device (2b) further includes a connecting port 8, such as a USB connecting port, mounted on the housing 21 and coupled to the processing unit 5. As shown in FIG. 17, the connecting port 8 is suitable for electrical connection with an external electronic device 300, such as a notebook computer, by means of a connecting wire 301 such that the circulatory information can be outputted from the processing unit 5 to the external electronic device 300 through the connecting port 8 and the connecting wire 301 for recording purposes.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. An electrical nerve stimulator comprising:
a current generator for generating a current signal; and
a main probe device connected electrically to said current generator for receiving the current signal therefrom, and including
a housing having a tubular end portion,
a probe disposed in said housing, and having a tip extending outwardly of said end portion of said housing, and
a cover mounted detachably on said end portion of said housing and having a flexible absorbent cap portion for covering said tip of said probe;
wherein said main probe device is operable so as to generate a stimulating signal based on the current signal from said current generator and to output the stimulating signal through said probe.

2. The electrical nerve stimulator as claimed in claim 1, wherein said absorbent cap portion of said cover is made of a cotton material.

3. The electrical nerve stimulator as claimed in claim 1, wherein said cover is connected threadedly to said end portion of said housing.

4. The electrical nerve stimulator as claimed in claim 1, wherein:
said end portion of said housing has an annular outer surface formed with at least one protrusion; and
said cover further has a sleeve portion connected to said absorbent cap portion, sleeved on said end portion of said housing, and having an annular inner surface formed with at least one first positioning groove that engages releasably said protrusion of said end portion of said housing, thereby positioning said cover on said end portion of said housing.

5. The electrical nerve stimulator as claimed in claim 4, wherein:
said sleeve portion of said cover has a rim flange;
said main probe device further includes an actuator mounted movably on said housing, and having an operating end portion, and a pushing end portion opposite to said operating end portion, and pushing said rim flange of said sleeve portion of said cover in response to movement of said operating end portion to move said cover away from said end portion of said housing such that said protrusion of said open end of said housing disengages said first positioning groove in said sleeve portion of said cover.

6. The electrical nerve stimulator as claimed in claim 5, wherein:
said annular inner surface of said sleeve portion of said cover is further formed with a second positioning groove;
said end portion of said housing is defined by an annular wall that is formed with a through hole corresponding to said second positioning groove; and
said main probe device further includes a driving rod disposed in said housing, and having a connecting end connected to said operating end portion of said actuator such that said driving rod is movable with said operating end portion of said actuator, and an engaging end opposite to said connecting end, extending outwardly of said through hole in said end portion of said housing, engaging releasably said second positioning groove in said sleeve portion of said cover, and moving away from said second positioning groove in said sleeve portion of said cover in response to movement of said operating end portion of said actuator to thereby disengage said second positioning grove in said sleeve portion of said cover.

7. The electrical nerve stimulator as claimed in claim 1, wherein said current generator separates from said housing of said main probe device, is wiredly-connected to said main probe device, and is detachable from said main probe device.

8. The electrical nerve stimulator as claimed in claim 7, further comprising an auxiliary probe unit wiredly-connected to said current generator, and including
a handle body having a tubular end portion,
a probe disposed in said handle body, and having a tip extending outwardly of said end portion of said handle body, and
a cover mounted detachably on said end portion of said handle body and having a flexible absorbent cap portion for covering said tip of said probe of said auxiliary probe unit.

9. The electrical nerve stimulator as claimed in claim 1, wherein said main probe device further includes:
an amplitude adjusting unit connected electrically to said current generator for receiving the current signal therefrom, including a control member mounted on said housing, and adjusting amplitude of the current signal in response to operation of said control member so as to generate the stimulating signal; and
a switch mounted on said housing, connected electrically between said amplitude adjusting unit and said probe, and operable between an ON-state, where the stimulating signal is outputted from said amplitude adjusting unit through said switch and said probe, and an OFF-state, where output of the stimulating signal is terminated.

10. The electrical nerve stimulator as claimed in claim 9, wherein said main probe device further includes:
a processing unit disposed in said housing, coupled to said amplitude adjusting unit and to a common node between said switch and said probe, receiving the stimulating signal from said amplitude adjusting unit so as to generate amplitude information associated with the stimulating signal, and calculating duration of the ON-state of said switch based on detection of output at said common node so as to generate therapy duration information associated with the duration of the ON-state of said switch; and
a display unit mounted on said housing, and coupled to said processing unit for displaying the amplitude information and the duration information thereon.

11. The electrical nerve stimulator as claimed in claim 10, wherein:
said main probe device further includes a temperature sensor disposed in said housing, coupled to said processing unit, and sensing temperature of said probe so as to output an output signal associated with the temperature of said probe to said processing unit;
said processing unit receives the output signal from said temperature sensor so as to generate temperature information corresponding to the temperature of said probe and displayed on said display unit.

12. The electrical nerve stimulator as claimed in claim 10, wherein said main probe device further includes a connecting port mounted on said housing, coupled to said processing unit, and adapted to be wiredly-connected to an external electronic device.

13. The electrical nerve stimulator as claimed in claim 10, wherein said main probe device further includes said current generator, and said current generator is disposed in said housing.

14. The electrical nerve stimulator as claimed in claim 13, further comprising an auxiliary probe unit wiredly-connected to said processing unit of said main probe device, and including
a handle body having a tubular end portion,
a probe disposed in said handle body, and having a tip extending outwardly of said end portion of said handle body, and
a cover mounted detachably on said end portion of said handle body and having a flexible absorbent cap portion for covering said tip of said probe of said auxiliary probe unit.
